(19) United States
(12) Patent Application Publication
(10) Pub. No.: US 2010/0022818 A1
(43) Pub. Date: Jan. 28, 2010

(54) Applicators for brachytherapy treatment

(76) Inventors: Steve Axelrod, Los Altos, CA (US); Thomas W. Rusch, Hopkins, MN (US); Linda Kelley, Los Gatos, CA (US); Lucymarie Mantese, Palo Alto, CA (US)

Correspondence Address: Thomas M. Freiburger, P.O. Box 1026, Tiburon, CA, 94920, US

(21) Appl. No.: 12/220,717

(22) Filed: Jul. 25, 2008

(51) Int. Cl. A61M 36/04

(52) U.S. Cl. 600/3

(57) ABSTRACT

An applicator for brachytherapy radiation avoids over-radiation in the distal axial direction from a source positioned in the applicator, by attenuating distal axial radiation which tends to over-radiate, causing a “hot spot” in tissue in the axial direction, when the radiation travels through air space in the lumen.

### BACKGROUND OF THE INVENTION

This invention concerns radiation therapy, especially electronic x-ray brachytherapy, for treating tissues which may have diffuse proliferative disease.

In brachytherapy, a radiation source (or a plurality of sources) is generally placed within a surgically created or naturally occurring cavity in the body. Such therapy often follows surgical treatment of cancer. The recently developed and now preferred radiation source for use with this invention is a miniature electronic x-ray source. Alternatively, selected low-energy radio-isotopes may be used with commensurate safety measures. One advantage of these sources, particularly the x-ray sources, is that their absorbed dose intensity can be conveniently manipulated with regard to output, on/off or modulated, and they can easily be attenuated locally.

Before the availability of miniature x-ray tubes, most brachytherapy was performed using high-energy isotopes like Iridium 192. Such high-energy isotopes are not capable in general of local attenuation because within the distances of interest for brachytherapy, their intensity will be basically unaffected by all practical attenuators.

With conventional intracavitary brachytherapy, a therapist prescribes a therapeutic dose of radiation to be administered to a volume of tissue (the target tissue) lying just outside the treatment cavity into which the radiation source or sources will be placed.

Generally the prescribed dose will specify a uniform minimum dose to be delivered at a preferred depth outside the treatment cavity (the prescription depth). Because by the laws of physics, radiation intensity falls off more or less exponentially with increasing distance from the radiation source, the prescription will include, either explicitly or implicitly, a maximum dose above which normal tissue may be subject to substantial necrosis. It is therefore generally desirable to create and maintain a predetermined space between the source of radiation and the first tissue surface to be treated (generally the cavity surface) in order to moderate the surface dose while still delivering the prescribed dose at the prescription depth.

This is usually accomplished by placing an applicator in the cavity which both fills and shapes the cavity into, most often, a solid figure of revolution (e.g., generally a sphere, cylinder or ellipsoid), and positions the radiation source along a central axis of the cavity so formed and along which the source may be traversed. It is common for the prescription to be delivered from several positions or dwell points along the axis of the applicator, often starting at the distal-most position, and sequentially withdrawn through the more proximal positions.

Some applicators are solid and of a polymer such as Noryl (GE Plastics), Radel (Solvay Advanced Polymers), or Ultem (GE Plastics), or comprising a composite structure of polymers, and of a shape to establish the necessary distances to effect proper delivery of the prescription (as in application Ser. No. 12/075,120, filed Mar. 5, 2008, which is incorporated herein by reference) while others have an inflatable element like a balloon (see, e.g., application Ser. No. 10/683,885, filed Oct. 10, 2003, incorporated herein by reference), which may be inflated to shape the cavity. It is preferable that the material of the solid applicator is substantially matched to attenuation of soft tissue. This simplifies treatment planning. If the applicator has a thin balloon such that attenuation due to the balloon may be substantially ignored, the balloon is preferably inflated using a fluid medium which has radiation attenuation properties similar to those of tissue. Water is such a medium, and again, this choice of medium simplifies treatment planning.

Traditionally, the radiation source is introduced into the applicator through a lumen positioned at a predetermined location in relation to the outer surface of the applicator (the inflated outer surface if of a balloon type). This location is usually along a central axis of the applicator, for example a lumen in the case of a solid type, or within a tubular source guide if a balloon type. These lumina are generally open at their proximal end to facilitate source insertion, but closed at their distal end.

As the source is withdrawn, an air gap is formed distally of the source within the lumen being traversed. The radiation attenuation of air is substantially less than that of the applicator material (or inflation medium if of a balloon type) so as to be near zero attenuation. Therefore, since the radiation distally down the applicator is only minimally attenuated, there is a radiation contribution from every sequential dwell point during the treatment, and a “hot spot” may be generated distal of the applicator lumen as the source is traversed away from its distal-most position.

It is an objective of this invention to overcome such hot spots in brachytherapy applicators. Other objectives will become apparent from the following discussion and figures.

### SUMMARY OF THE INVENTION

We have discovered that an effective method to eliminate central hot spots distal of conventional brachytherapy applicators during radiotherapy is to incorporate a disc of attenuating material covering, and preferably somewhat overlapping the distal end of the central applicator lumen. A preferred disc is of Noryl (a blend of polyphenylene oxide and polystyrene) doped with Barium Sulfate (BaSO₄). The overlap is preferable since the radiation “jets” from the more proximal dwell points tends to spread with increasing distance.

Overlapping is not required to achieve substantial results using the concept of the invention, however. With electronic radiation sources, the lumen might be on the order of 6 mm in diameter. A preferred disc would then be on the order of 7 mm diameter. The disc thickness would be dependent on loading and the resulting attenuation, but would be sized to produce the prescribed distal dose cumulatively considering the contribution from all dwell points. An example might be a Noryl disc of 2 mm thickness with a 6% preferred loading of BaSo₄by weight.

In another embodiment water is used as a radiation attenuator in the distal axial direction. The water can be pure or a solution of selected compounds, such as one or more salts.

Other disc materials and loadings will occur to those of skill in the art and are to be considered within the scope of the invention. Present applicators have been largely developed for use with high-energy isotope sources and do not include axial attenuators.

### DESCRIPTION OF THE DRAWINGS

FIG. 1 depicts a solid applicator of the invention with an attenuating disc in a counterbore in the distal tip of the applicator, and a miniature x-ray tube on the distal end of a source catheter within the central lumen.

FIG. 2 depicts a balloon applicator of the invention with an attenuating disc bonded to the applicator source guide, covering and overlapping the source guide lumen, and a miniature x-ray tube on the distal end of a source catheter within the central lumen.

FIG. 3 shows a modified solid applicator as in FIG. 1 with a different form of attenuation.

FIG. 4 shows a modified balloon applicator as in FIG. 2 with a different form of attenuation.

### DESCRIPTION OF PREFERRED EMBODIMENTS

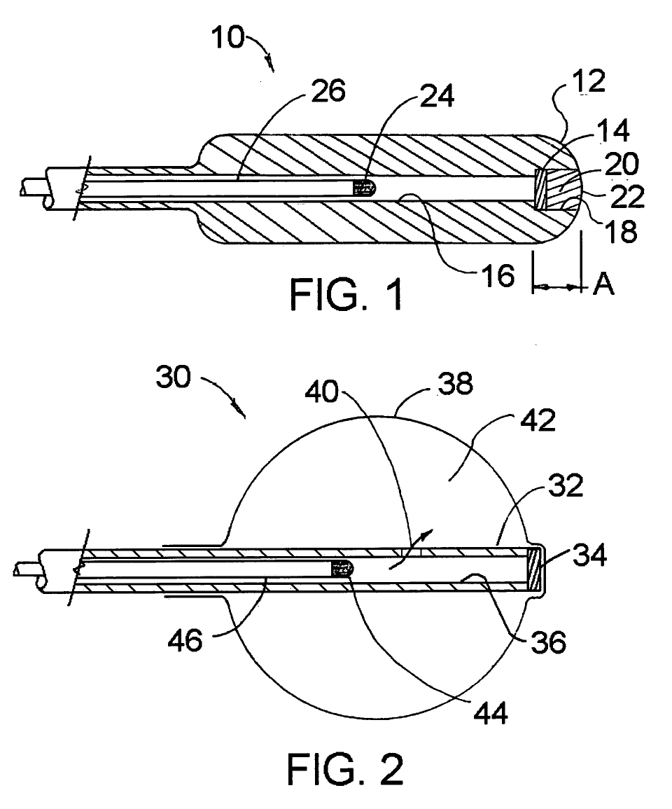

FIG. 1 shows a solid, endometrial applicator 10 having an applicator body of generic shape with a counterbored tip 12 into which has been fitted an attenuating disc 14, preferably of Noryl loaded with BaSO₄. The counterbore diameter is greater than the diameter of the lumen 16 in this preferred embodiment, and the tip counterbore 18 distal of the disc 14 has been filled in a manner so as to secure the disc in the tip. Bonding a plug 20 into the counterbore 18 is one method, but a screwed plug could also be used (not shown). Still other traditional methods will occur to those of skill in the art. Preferably the outer surface 22 of the filler or plug conforms to the overall outer applicator shape desired.

The disc 14 is aligned with the lumen 16 and with axially emitted radiation emanating from the lumen. The disc thickness and BaSO₄ filling level are adjusted such that the cumulative dose delivered distally from each dwell point is commensurate with the prescription dose. The disc diameter is preferably, though not necessarily, from 10% to 20% greater than the lumen diameter, making necessary the size of the counterbore. A radiation source 24 on the end of a source catheter 26 is shown within the lumen 16.

The preferred solid applicator embodiment is as described above, but other variations or designs of solid applicators can be improved by applying similar methods to those described above. For example, multi-lumen solid applicators can be fitted with separate discs beyond the end of and in line with each lumen, or collectively fitted with a larger, single attenuating disc overlapping all lumina. Also, the attenuating disc can be of different sizes, even of diameter equal to the lumen, if desired, although slightly larger is preferred for reasons explained above. Further, the attenuating disc may comprise simply a plug such as the plug 20 bonded into a bore 18 in the end of the solid applicator, and being of greater attenuating property than the solid applicator, in lieu of the two-piece arrangement 14, 20 shown in FIG. 1.

FIG. 2 depicts a preferred balloon applicator 30 of substantially generic design, but where the distal end of the applicator's source guide 32 has an attenuating disc 34 bonded to and overlapping the diameter of the source guide lumen 36. The distal end of the balloon 38 encompasses and is bonded to the disc 34 in this embodiment, the disc having greater attenuating properties than the shaft and the balloon. Fluid flow to inflate the balloon 38 is shown by an arrow passing through a port 40 into the balloon volume 42. A radiation source 44 is shown within lumen 36 positioned at the end of a source catheter 46.

The incorporation of an attenuating disc at the end of the lumen 36 in a balloon applicator can be accomplished in several ways. The balloon itself can be doped in a preferably circular area at a spot surrounding the distal end of the lumen, i.e. the end of the source guide shaft or body 32 which forms the lumen. See copending applications Ser. Nos. 10/683,885, 10/962,247 and 11/471,013), in which selected areas of balloons are doped with attenuating materials in balloon applicators. The balloon thickness can be a limiting factor as to whether sufficient attenuating material can be incorporated in the balloon, but this will reduce the “hot spot” effect. The shaft 32 itself could be fitted with a cap over its end, the cap having attenuating material, or, in cases in which the diameter of the attenuating element is deemed sufficient at the same diameter as the lumen 36, a plug having attenuating material can be fitted into the lumen at the end of the shaft 32. Other methods and apparatus are also possible.

Other balloon applicator designs may be substituted for that shown, including incorporation of other functionality, for example drainage, with appropriate conventional details of construction as would be apparent to those of skill in the art.

With either embodiment, the distal axial dose can be engineered to deliver the prescribed dose throughout the target tissue without a hot spot discontinuity centrally at the distal tip of the applicator. For example, we have found that using solid applicators with outer diameters according to the table below, a concentric inner lumen of 5.6 mm and disc outer diameters of 6.6 mm, a conformal dose of radiation can be generated from those disc thicknesses corresponding in the table.

| Solid Applicator of | Noryl Disc with 6% |
|---|---|
| Outer Diameter, mm | BaSO₄ Thickness, mm |
| 20 | 1 |
| 25 | 2 |
| 30 | 2 |
| 35 | 3 |

A conformal dose of radiation has isodose surface shapes that mimic the outer surface of the applicator. Isodose surfaces are imaginary surfaces where the dose intensity is uniform over the entire surface. It is a simple matter to determine disc dimensions for balloon conformal doses once the balloon shapes are selected.

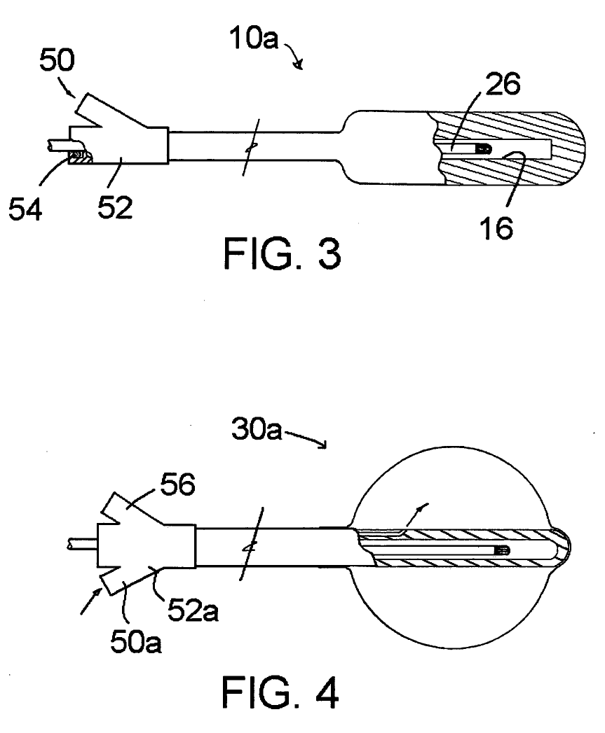

FIG. 3 shows a variation of the device of FIG. 1, wherein an applicator 10a has a water port 50 on its proximal end hub 52. The source catheter 26 passes through the hub and is sealed, as indicated, with an appropriate form of seal 54 such as an O-ring. The water port 50 admits water to the lumen 16, so that water contained in the lumen beyond the distal end of the source catheter 26 acts to attenuate the radiation from the source in the distal axial direction. The water (normally saline or another salt solution) can be used or it can be used in combination with the disc or plug attenuator shown in FIG. 1 to achieve a desired attenuation and eliminate hot spots. Water (saline) is a good attenuator because it is similar in attenuating properties to the applicator body. By eliminating air from the lumen distal of the source 24, the hot spot problem is essentially eliminated from the treatment plan calculation. Because the position of the source catheter 26 will cause different degrees of displacement of the water within the lumen, the filling port 50 can be connected to a water source in which water can be pushed back when water is displaced.

FIG. 4 shows a similar arrangement for a balloon applicator 30a. The hub 52a has a filling port 56 for the balloon and a filling port 50a for the lumen, for providing attenuating liquid to attenuate in the distal axial direction.

Although the description above assumes an electronic radiation source such as the preferred miniature x-ray tube of, for example, U.S. Pat. Nos. 6,319,188 and 7,158,612, incorporated entirely herein by reference, the radiation source (or sources) could equally comprise low-energy isotopic seeds of Iodine 125 or Palladium 103 mounted on a wire or wires (not shown).

From the description of preferred embodiments above, other embodiments will be apparent to those of skill in the art and are to be considered as within the scope of this invention as defined in the following claims.

We claim:

1. A brachytherapy applicator configured to reduce or eliminate over-radiation in the distal axial direction, comprising:
an essentially solid applicator body, and at least one lumen inside the applicator body, of a size to receive a source of radiation inserted therein for brachytherapy radiation of tissue surrounding the applicator when placed into a patient's tissue, and
an attenuator generally at the distal end of the lumen, in line axially with the lumen, the attenuator having attenuating properties so as to reduce or eliminate over-radiation in the distal axial direction.

2. The applicator of claim 1, wherein the attenuator is positioned within a recess at the distal end of the applicator, the recess and the attenuator being larger in diameter than the lumen.

3. The applicator of claim 2, further including a closure element in the recess distal of the attenuator.

4. The applicator of claim 3, wherein the attenuator is a disc, and wherein the closure element comprises a plug distal of the disc, closing the recess.

5. The applicator of claim 2, wherein the attenuator is a plug closing the recess.

6. The applicator of claim 1, wherein the applicator body is formed of material having generally similar attenuation properties to living tissue within which the applicator is to be used.

7. The applicator of claim 1, wherein the attenuator includes barium sulfate.

8. The applicator of claim 1, wherein the attenuator comprises water or water solution within the lumen so as to fill the lumen distal of a radiation source when a radiation source is inserted.

9. The applicator of claim 1, wherein the attenuator comprises a liquid within the lumen so as to fill the lumen distal of a radiation source when a radiation source is inserted.

10. The method of claim 9, wherein the applicator has a proximal end hub with a filling port for admitting liquid into the lumen.

11. A brachytherapy applicator configured to reduce or eliminate over-radiation in the distal axial direction, comprising:
a balloon applicator comprising an inflatable balloon secured to a shaft having an internal lumen, the internal lumen being sized to a receive a source of radiation inserted therein for brachytherapy radiation of tissue surrounding the balloon applicator when placed into a patient's tissue, and
a radiation attenuator generally at the distal end of the shaft's lumen and in line axially with the lumen, the attenuator having attenuating properties so as to reduce or eliminate over-radiation in the distal axial direction.

12. The applicator of claim 11, wherein the attenuator comprises a disc at the distal end of the shaft.

13. The applicator of claim 10, wherein the attenuator comprises a cap over the distal end of the shaft.

14. The applicator of claim 11, wherein the attenuator comprises a liquid within the lumen so as to be positioned distal of a radiation source when a radiation source is inserted.

15. The applicator of claim 14, wherein the applicator has a proximal end hub with a filling port for admitting liquid into the lumen.

16. An applicator for brachytherapy radiation treatment of a living patient, with provision for reducing or eliminating over-radiation of tissue in the distal axial direction, comprising:
the applicator having a body forming an internal lumen within the applicator, the internal lumen being sized to receive a source of radiation inserted therein for brachytherapy radiation of tissue surrounding the applicator when placed into a patient's tissue,
the internal lumen being essentially linear such that, with the source of radiation emitting radiation from a series of different positions along the linear lumen, over-radiation ordinarily tends to occur in the distal axial direction, and
a radiation attenuating device generally at the distal end of the lumen and aligned with the lumen.

17. The applicator of claim 16, wherein the attenuating device comprises a disc positioned at the distal end of the lumen.

18. The brachytherapy applicator of claim 16, wherein the applicator is essentially solid and comprises said body forming the lumen, and the body including at a distal end a recess, with the attenuating device positioned in the recess.

19. The brachytherapy applicator of claim 18, wherein the recess and the attenuating device are larger in diameter than the lumen.

20. The brachytherapy applicator of claim 16, wherein the attenuating device comprises a liquid within the lumen so as to fill the lumen distal of a radiation source when a radiation source is inserted.
